(19) United States
(12) Patent Application Publication
(10) Pub. No.: US 2007/0223714 A1
(43) Pub. Date: Sep. 27, 2007

(54) OPEN-AIR NOISE CANCELLATION SYSTEM FOR LARGE OPEN AREA COVERAGE APPLICATIONS

(76) Inventor: Masao Nishikawa, La Jolla, CA (US)

Correspondence Address: INGRASSIA FISHER & LORENZ, P.C., 7150 E. CAMELBACK, STE. 325, SCOTTSDALE, AZ, 85251, US

(21) Appl. No.: 11/624,468

(22) Filed: Jan. 18, 2007

Related U.S. Application Data
(60) Provisional application No. 60/760,083, filed on Jan. 18, 2006.

(51) Int. Cl. A61F 11/06

(52) U.S. Cl. 381/71.1

(57) ABSTRACT

A variety of open-air noise cancellation systems are disclosed. The systems are configured to suit the needs of the particular application, for example, an open-air sound wall installation, an open-air enclosure for a quiet area, or a window/door treatment application. A particular system may a digital-based processing architecture having a digital power amplifier that shares a common circuit board. The processing architecture receives noise signals, processes out-of-phase noise cancellation signals in response to the noise signals, and generates out-of-phase sound waves that effectively cancel low frequency components of the noise signal. One system variation utilizes passive sound absorbing blades or shutters to reduce high frequency components of the noise signal. The blades can be installed as a door or window shutter mechanism.

### RELATED APPLICATION

This application claims the benefit of U.S. provisional patent application Ser. No. 60/760,083, filed Jan. 18, 2006.

### TECHNICAL FIELD

The present invention relates generally to environmental noise control systems. More particularly, the present invention relates to an open-air noise cancellation system suitable for large open area applications such as patio covers, open restaurant areas, open doors, and open entrance areas.

### BACKGROUND

Environmental noise has become a very significant issue for many homes, businesses and other institutions. A variety of different factors contribute to the problem of environmental noise pollution. They include increasing population density, per capita space reduction, and increasing levels of industrial, transportation and residential noise.

Common noise sources include roads and freeways, airplanes, industrial institutions, plants and factories, air conditioners and pool equipment, and many others.

According to the United States Environmental Protection Agency and a host of other government and not-for-profit institutions, noise pollution is a significant environmental concern and may cause a variety of significant problems. For example, people exposed to transportation noise may experience such consequences as loss of sleep, productivity loss, hearing problems, loss of physical well-being, stress, and increasing health care costs.

Property values may also be lowered because of nearby transportation noise sources.

Accordingly, it is desirable to have systems, devices, and apparatus for reducing environmental noise. Furthermore, other desirable features and characteristics of the present invention will become apparent from the subsequent detailed description and the appended claims, taken in conjunction with the accompanying drawings and the foregoing technical field and background.

### BRIEF SUMMARY

A system is provided for reducing the effects of environmental noise by actively canceling noise which is directly and indirectly approaching the noise reduction target area (the “protected” side). The system reduces the amount of sound traveling through the noise reduction system, thus reducing the amount of environmental noise heard on the “protected” side of the area. The example embodiment of the system has multiple microphones and speakers. A noise collection microphone located forward (or outside) the noise reduction target area detects and provides accurate information on the noise elements such as frequency and power of the environmental noises. Then the noise information from the microphone is electronically processed to provide signals having the opposite phase of the noise signals. The out-of-phase signals are transferred to amplifiers for output to the speakers for the same amount of sound simply in opposite phases to cancel the original noises. Then, error correction microphones located at the noise reducing space detect the delta (difference) between the original noise level for the space and the out-of-phase signal output from the speaker. Such information is continuously fed back to the active noise cancellation unit for continuous adaptation and corrections. For example, a positive difference signal may indicate too little cancellation, while a negative difference signal may represent too much cancellation. The feed back is operated in multiple locations of the microphones.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a graph that depicts noise reduction characteristics of a barrier wall versus distance for different frequencies;

FIG. 2 is a diagram of a noisy environment that includes diffracted sound;

FIG. 3 is a diagram of a noisy environment having a noise protection enclosure;

FIG. 4 is a diagram of a noisy environment with an embodiment of an active noise cancellation system;

FIG. 5 is a schematic representation of a digital implementation of an embodiment of a noise cancellation system;

FIG. 6 is a schematic representation of an implementation of a noise cancellation system that utilizes a digital power amplifier;

FIG. 7 is a diagram that illustrates the placement of an embodiment of an active noise cancellation unit;

FIG. 8 is a schematic representation of an embodiment of a system having a plurality of active noise cancellation units assembled to form a protected area;

FIG. 9 is a schematic representation of another embodiment of a system having a plurality of active noise cancellation units;

FIG. 10 is a schematic front view of an embodiment of a door/window noise cancellation unit;

FIG. 11 is a perspective view of an embodiment of a window noise cancellation unit;

FIG. 12 is a schematic front view of an embodiment of a door/window noise cancellation unit that utilizes shutters;

FIG. 13 is a perspective view of an embodiment of a window noise cancellation unit that utilizes shutters; and

FIG. 14 is a graph of a typical noise characteristic of a jet aircraft before and after noise cancellation.

### DETAILED DESCRIPTION

The following detailed description is merely illustrative in nature and is not intended to limit the embodiments of the invention or the application and uses of such embodiments. Furthermore, there is no intention to be bound by any expressed or implied theory presented in the preceding technical field, background, brief summary or the following detailed description.

Various techniques, technologies, and methodologies may be described herein in terms of functional and/or logical block components and various processing steps. It should be appreciated that such block components may be realized by any number of hardware, software, and/or firmware components configured to perform the specified functions. For example, an embodiment of the invention may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that the present invention may be practiced in conjunction with any number of noise cancellation applications and that the open window, patio, and door systems described herein are merely example applications for the invention.

For the sake of brevity, conventional techniques related to audio signal processing, digital signal processing, filtering, noise cancellation, and other functional aspects of the systems (and the individual operating components of the systems) may not be described in detail herein. Furthermore, the connecting lines shown in the various figures contained herein are intended to represent example functional relationships and/or physical couplings between the various elements. It should be noted that many alternative or additional functional relationships or physical connections may be present in a practical embodiment.

The following description may refer to elements or features being “connected” or “coupled” together. As used herein, unless expressly stated otherwise, “connected” means that one element/feature is directly joined to (or directly communicates with) another element/feature, and not necessarily mechanically. Likewise, unless expressly stated otherwise, “coupled” means that one element/feature is directly or indirectly joined to (or directly or indirectly communicates with) another element/feature, and not necessarily mechanically. Thus, although the schematics shown in the figures depict example arrangements of elements, additional intervening elements, devices, features, or components may be present in an actual embodiment (assuming that the functionality of the system is not adversely affected).

In general, when there are environmental noise issues, sound barrier walls, transparent glass panel walls, or complete enclosures such as window covered patios are installed between the source and the receiver of the noise. The height, location, and the materials of the sound walls play a significant role in determining the effectiveness of the sound walls. In general, the closer the wall is placed to the sound source, or to the receiver, the better the noise reduction effect. The higher the wall, the better the effect would be for noise reduction. However, there are many height limitations in installing walls, and making the walls too high reduces brightness of the space and increases psychological pressures. Also, high glass walls and complete enclosure walls using glass or other transparent material are used to cut the noise for specific locations, but at the same time such remedies cut the air breeze and reduce outdoor airflow. For buildings, the basic way to cut the noise is to close the windows or doors, thus the fresh air flow is also restricted.

There are two practical measurement criteria in grading the materials and effectiveness of the sound walls. Transmission Loss (or Sound Transmission Class=STC) is the sound energy transmitted through the wall from the sound source to the receiver when the wall is installed on the line of sight point. Usually, high Transmission Loss of approximately 30 dBA is considered to be a good sound barrier. Absorption Ratio (or Noise Reduction Co-efficiency=NRC) is the other criteria to determine how much of the sound energy is absorbed (and reflected) by such sound barriers. For example, a wall with a 0.90 rating means that 90% of the noise is absorbed, and 10% is reflected.

These criteria are good measurement criteria for the sound walls, however, there is another important path called the “diffraction path” where sound travels from the source to the receiver around the sound barrier. Diffraction is a physical phenomena where any waves, whether light, sound, or water travels around an object (as if the waves bend around the object). In the case of a vertically deployed sound wall, or blades in the case of window shutters and similar objects, sound bends at the top of the wall or at the edges of the blades traveling towards the receive point of the sound. Other than direct sound paths, diffraction is one of the most significant paths of sound that can travel from the source to the receiver in an open air environment.

The diffraction amount depends on the length of the wave as well as the angles from the source through the object to the receiver. Sound with longer wavelength (lower frequency sound) diffracts more, and sound with shorter wavelength (higher frequency sound) diffracts less. When there is a wall between the noise source and the receiver of the noise, the more angles the noise has to travel over the wall to the receiver on the other side of the wall, the less noise diffracts and the less noise reaches to the receiver. In other words, the higher the wall, the less the receiver hears the noise. Audible sound frequencies are between 20 Hz to 20,000 Hz, which corresponds to wavelengths between 17 mm up to 17 m. Sound travels at the speed of 340 meters per second, thus a frequency of 100 Hz corresponds to a 3.4 m wavelength, and a frequency of 1,000 Hz corresponds to a 34 cm wavelength.

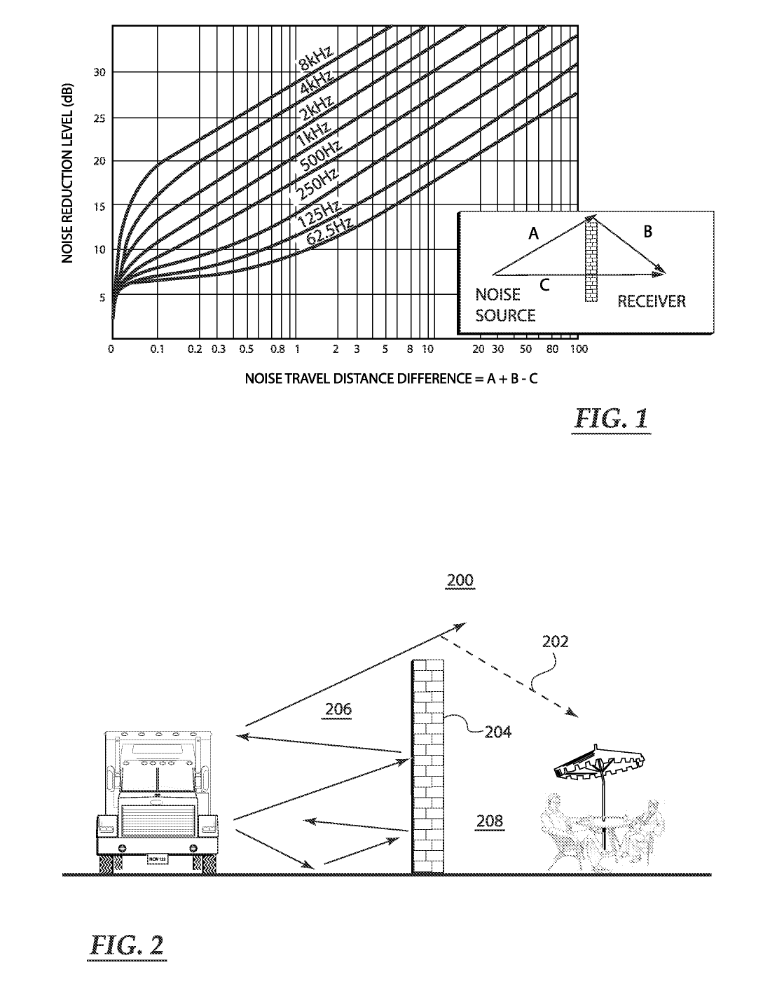

FIG. 1 is a graph that depicts noise reduction characteristics of a barrier wall versus distance for different frequencies. The vertical scale represents the noise reduction level in dB, and the horizontal scale represents the noise travel distance difference (in meters). This difference, a+b−c, is based on the sound path distances for a simple sound barrier wall, as depicted in the lower right portion of FIG. 1. As illustrated in FIG. 1, higher frequencies of noise require less height (difference in sound travel path) of a sound barrier wall to reduce noise levels, whereas for lower frequencies noise reduction does not become significant until the sound barrier wall reaches a particular height. This relationship between the height and difference in sound travel path and the amount of noise diffracted in relation to the frequencies of such noise in the barrier wall case illustrated in FIG. 1 also applies to the height of each of the multiple blades placed on the window and door shutters for the purposes of shutting noise coming through open windows and doors. When there are blades on the shutters, the diffracted noise is less when the height of the blades placed on the shutters is higher—higher blades result in less noise entering the protected area. The difference in the noise travel path is defined as “a+b−c” in the triangular figure illustrated in FIG. 1. The distance “c” is the direct sound path distance without the wall or without a blade. For an example, for noise of 2,000 Hz, in order to reduce the noise by 15 dB, it requires about 0.15 meters difference between the “a+b” and the “c.” Whereas for noise of 250 Hz, in order to achieve a 15 dB reduction, it requires about 1.25 meter more travel path, which means a higher wall.

The understanding of sound, reflection, absorption, and diffraction has been increased in the recent years and many improvements in the sound walls have been implemented. However, traditional applications have not addressed diffraction patterns for purposes of diffraction control to effectively reduce the unwanted noise. Also, the combination of acoustic and active noise cancellation technologies has not been utilized together.

An apparatus or system as described herein provides effective methods of reducing both the direct path and the diffraction path of environmental noise. In the practical embodiment, the mechanics and the electronics of the structure is composed of microphones and speakers using active noise cancellation techniques, as well as porous acoustic materials to absorb noise. The system is utilized to reduce noise approaching and going through components of the system. A system according to the invention allows the arrangement to maintain the light and the air breeze flow into the noise reduction target area while reducing the wide range of frequencies of the environmental noise.

FIG. 2 is a diagram of a noisy environment 200 that includes diffracted sound 202 traveling over a sound barrier wall 204, which may be a glass panel. FIG. 2 illustrates how, in normal environments, sound on one side 206 of the wall 204 travels over the wall 204 and how some of the sound is diffracted downward such that it travels to the receiver located on the other side 208 of the wall 204. The lower the frequency of the sound, the more diffraction occurs.

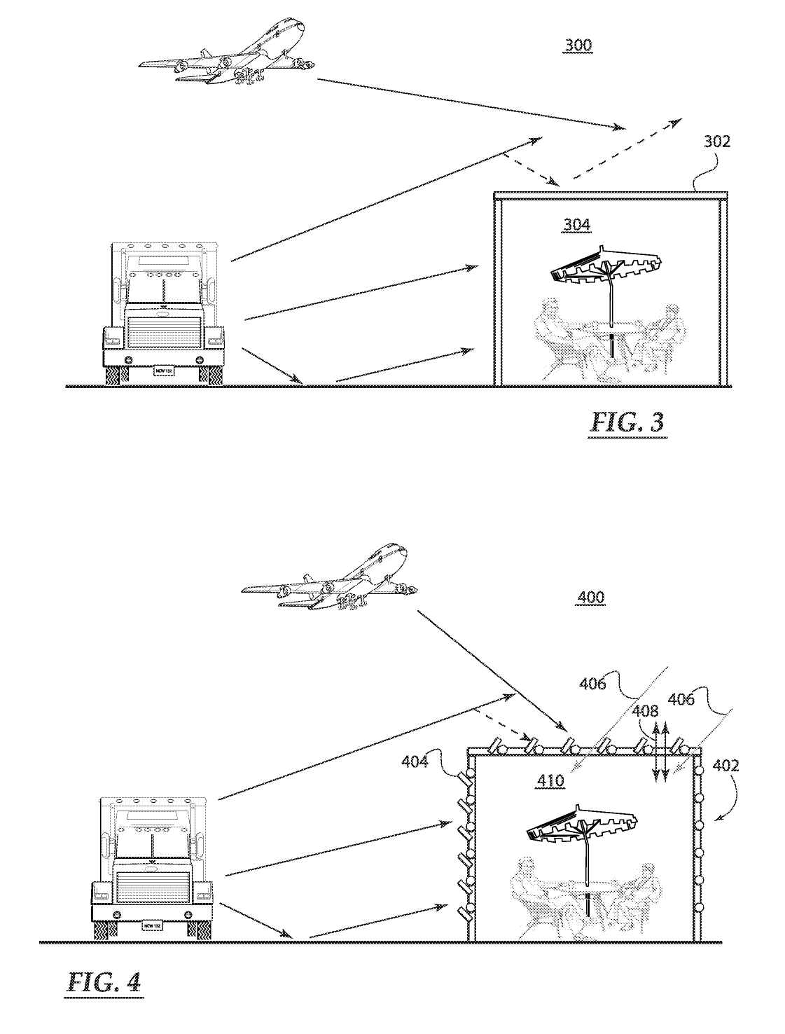

FIG. 3 is a diagram of a noisy environment 300 having a noise protection enclosure 302 that is intended to “surround” and isolate a protected area 304. Enclosure 302 may be formed of glass or other materials. FIG. 3 illustrates how the complete enclosure 302 cuts the noise but at the same time prevents flow of air and breeze to the receivers inside of the enclosure 302 and how the location is no longer an open area.

FIG. 4 is a diagram of a noisy environment 400 with an embodiment of an active noise cancellation system 402 deployed therein. This system 402 utilizes a combination of active noise cancellation components and diffraction control blades 404. The components can be mounted to an open frame structure that forms an “enclosure” for the protected quiet area. This open frame structure may include one or more barrier walls and/or an open-air canopy, cover, patio cover, roof, or ceiling as depicted in FIG. 4.

In FIG. 4, the circles represent noise cancellation speakers and the rectangles represent diffraction control blades 404. In the example embodiment of the present invention, the noise directly and indirectly approaching the system 402 is cancelled in part by the active noise reduction components, however, it is also cancelled by acoustic blade mechanisms 404 that are suitably configured to control the diffraction of the sound waves. FIG. 4 shows one practical implementation. Of the broad frequency range of an environmental noise (typically from 50 Hz to 2,000 Hz), the lower frequency portion of the noise, typically up to 700 Hz, is cancelled by the active noise cancellation elements, and over 700 Hz of mid to higher frequency noise is cancelled by the acoustic blade mechanisms 404. The dashed line in FIG. 4 depicts a diffracted sound wave, and how that diffracted sound is blocked by one of the blade mechanisms. The arrows 406 represent paths for the flow of light through open space, while the arrows 408 represent air flow paths that lead into the protected area 410.

Conventional active noise cancellation techniques leverage the so-called “closed air” and “feed back” environment. Such techniques are commonly used in headsets and cellular phones. In contrast, however, a system configured in accordance with the present invention applies to the open-air environment, and such a system may employ one or more of the following techniques, features, and aspects (without limitation): active noise cancellation techniques; diffraction control; output power level control; frequency characteristic and control; acoustic elements to control sound and noise; and open air optimization to offset open air noise.

In one example embodiment, a noise reduction system includes multiple sets of microphones and speakers. The microphones detect the noise, change the noise sound waves into noise cancellation electrical signals, and relay the noise cancellation signals to the speakers, which turn the signals back into sounds. The electronics create cancellation signals that are 180 degrees (within practical tolerances) out-of-phase with the actual noise signals. Thus, since the sounds from the speakers are of opposite phase from the noises, the generated sound actively cancels the unwanted noise sounds. The noise cancellation speakers add loud noises that are simply out of phase, and provides significant reduction of background noise.

A system according to the present invention provides effective methods and apparatus for implementing open-air noise cancellation for direct and diffracted noise control. The sound from the speakers is out-of-phase with the noise, thus canceling the noise sound. The noise cancellation speakers reproduce loud noises that are simply out-of-phase, thus performing significant reduction of background noise and producing a very quiet environment.

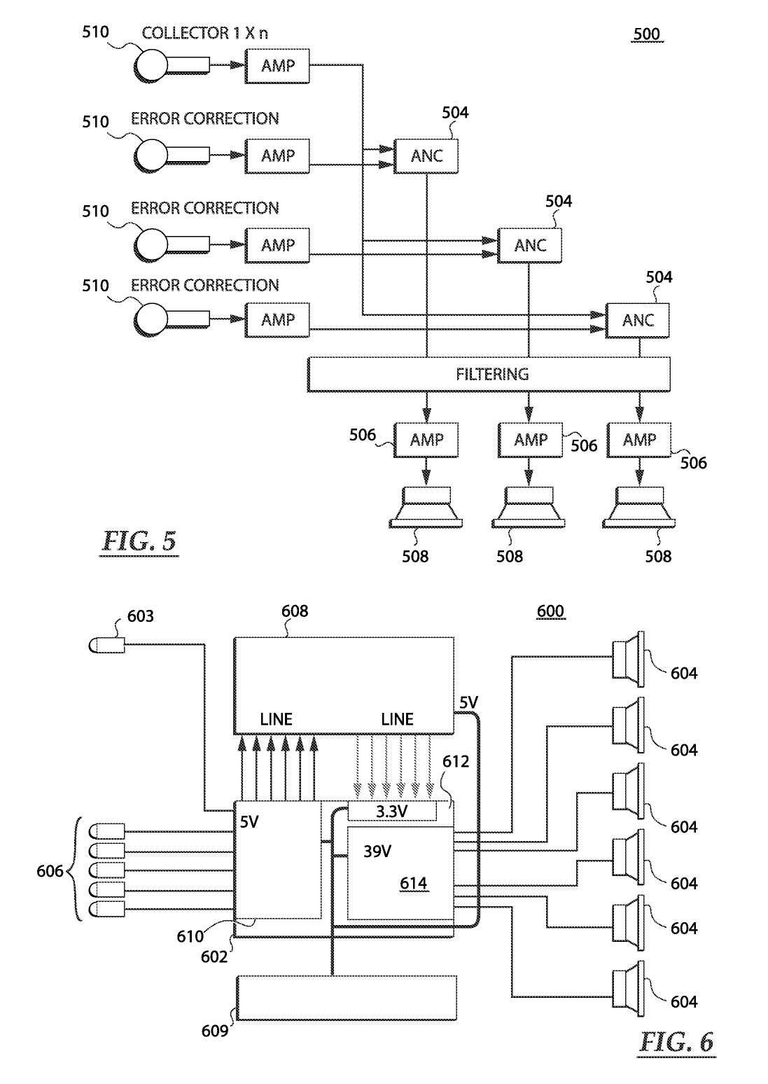

FIG. 5 is a schematic representation of a digital implementation of an embodiment of a noise cancellation system 500. This example digital system includes multiple sets of microphones and speakers. The noise collection microphone 502 located forward, or outside the noise reduction target area, detects and provides accurate information on the noise elements such as frequency, and power of the environmental noises. Then the noise information from the microphone 502 is electronically processed to provide signals having the opposite phase of the noise signals at one or more active noise cancellation controller (ANC) 504. The out-of-phase signals are transferred to amplifiers 506 for output to the speakers 508 for the same amount of sound simply in opposite phases to cancel the original noise. Then, the error correction microphones 510 located at the noise reducing space detect the delta (difference) between the original noise level reached to the space and the out-of-phase signals output from the speakers 508. Such “plus or minus” (too little cancellation, or too much cancellation signals) information is continuously fed back to the active noise cancellation controllers 504 for continuous adaptation and corrections. The feed back is operated in multiple locations of the microphones 510. In this manner, the processing architecture of the ANCs 504 are suitably configured to continuously adapt to optimize the respective noise cancellation signals.

In practice, an ANC 504 (or any given processing unit, processing architecture, or logical element described herein) may be implemented or performed with a general purpose processor, a content addressable memory, a digital signal processor, an application specific integrated circuit, a field programmable gate array, any suitable programmable logic device, discrete gate or transistor logic, discrete hardware components, or any combination thereof, designed to perform the functions described herein. A processor may be realized as a microprocessor, a controller, a microcontroller, or a state machine. A processor may also be implemented as a combination of computing devices, e.g., a combination of a digital signal processor and a microprocessor, a plurality of microprocessors, one or more microprocessors in conjunction with a digital signal processor core, or any other such configuration.

Moreover, the steps of a method or algorithm described in connection with the embodiments disclosed herein may be embodied directly in hardware, in firmware, in a software module executed by a processor, or in any practical combination thereof. A software module may reside in RAM memory, flash memory, ROM memory, EPROM memory, EEPROM memory, registers, a hard disk, a removable disk, a CD-ROM, or any other form of storage medium known in the art. In this regard, an exemplary storage medium can be coupled to a processor such that the processor can read information from, and write information to, the storage medium. In the alternative, the storage medium may be integral to the processor. As an example, the processor and the storage medium may reside in an ASIC. A practical ANC 504 may employ one or more processors and a suitable amount of memory in this manner to support its functionality.

FIG. 6 is a schematic representation of an implementation of a noise cancellation system 600 that utilizes a digital power amplifier 602. System 600 generally includes a noise collection microphone 603, speakers 604, error correction microphones 606, and one or more ANC units 608, as described above in the context of FIG. 5. FIG. 6 also depicts a power supply 609, which is suitably configured to provide various operating voltages to different components of system 600.

Digital power amplifier 602 is suitably configured to drive multiple loud speakers 604. System 600 is suitable for applications that require noise reduction in a wide area. Such applications may employ requires multiple speakers 604 and, consequently, high drive power. In this regard, digital power amplifier 602 efficiently reproduces the opposite sound waves for such wide area applications. For example, in order to drive six speakers 604 with 25 Watt amplifiers (total 150 Watts), digital power amplifier 602 uses only 170 Watts (approximately 90% efficiency) of power. In general, efficiencies of analog power amplifiers are low, thus they are not suitable for driving multiple speakers with multiple amplifiers for this application. On the other hand, regarding digital power amplifiers, for example, driving the six speakers (assuming 25 Watts each for a total output of 150 Watts) requires only 170 Watts of electric power supply (approximately 90% efficiency) by such amplifiers. In order to achieve wide area noise reduction with this system, practical embodiments will use high efficiency power amplifiers combined with other electronics described here.

In practice, the digital power amplifier 602 can be incorporated into the circuit board assembly used for the ANC unit 608, which is desirable to reduce the number of parts used in the system. In particular, this arrangement reduces the number of parts between the ANC unit 608 and the amplifier board, and allows system 600 to be manufactured in a simple and compact manner with a common circuit board for both the ANC unit 608 and the digital power amplifier 602.

For this embodiment, digital power amplifier 602 includes a microphone pre-amplifier 610 (which may be configured to operate with a 5-volt supply voltage). Pre-amplifier 610 obtains the measured sound signals, amplifies the signals, and provides the amplified signals as outputs to ANC unit 608. After processing, ANC unit 608 provides the processed noise cancellation signals to an analog-to-digital converter 612 (which may be configured to operate with a 3.3-volt supply voltage). Analog-to-digital converter 612 generates a digital input for an output stage 614. In an embodiment having a combined circuit, digital power amplifier 602 and ANC unit 608 are combined, thus eliminating the need for analog-to-digital and digital-to-analog conversion processes. Eventually, an output stage 614 generates amplified output signals that are utilized to drive speakers 604. Output stage may be configured to operate with a 39-volt supply voltage.

Further, an on-off switch for the power amplifiers can be implemented. In applications such as airplane noise that intermittently approach the noise reduction target area, it is only necessary to power the amplifiers when the noise level reaches a certain level, for an example, 55 decibels. Then, a suitably configured sensor automatically initiates power-up of the system 600. As soon as the noise source is further away and the noise level is reduced to 55 decibels (or less), the switch automatically shuts down the amplifiers.

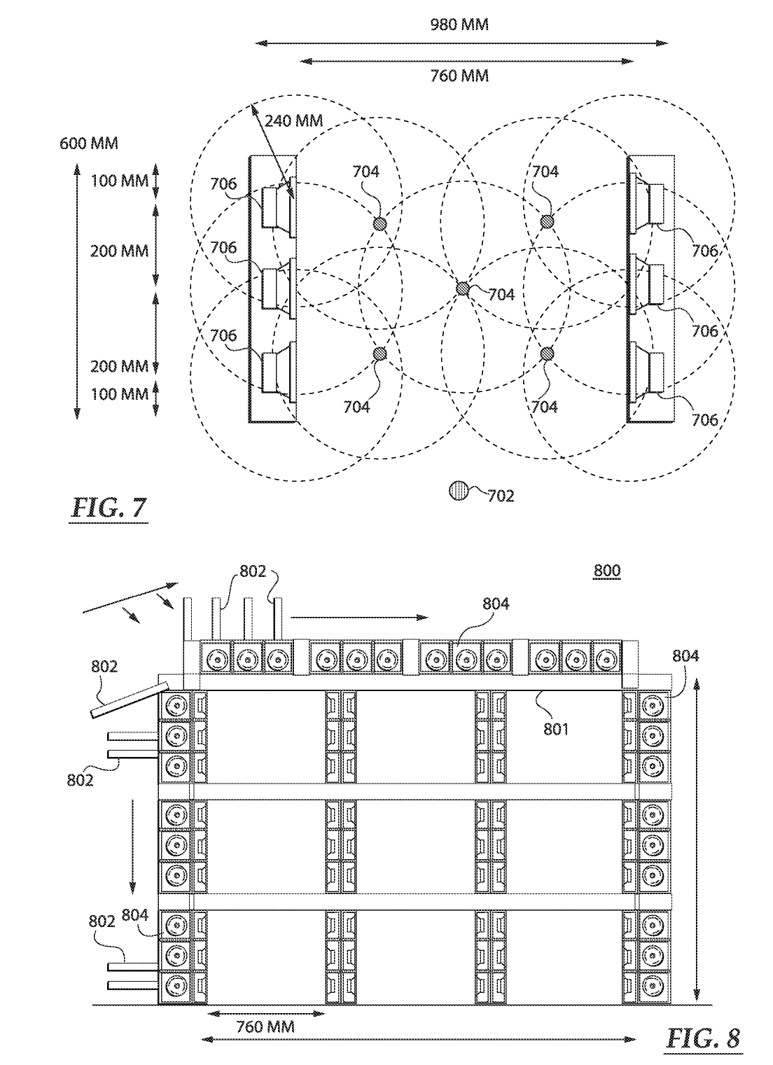

FIG. 7 is a diagram that illustrates the physical placement of an embodiment of an active noise cancellation unit. FIG. 7 represents a front or face view of an example implementation of one unit comprising multiple processing active noise cancellation elements. This particular active noise cancellation unit has one noise collection microphone 702 in the forward center, five error correction microphones 704, and six speakers 706 that are strategically located at the “open air wall surface” where the low frequency sounds are cancelled. In this view, noise comes from the forward location, first hits the noise collection microphone 702 which is shown at the bottom of the picture for convenience, but is actually located a distance (such as one meter) away from the face of the “open air wall surface.” The noise information captured by the noise collection microphone 702 is fed into all the active noise cancellation control electronics as shown in FIG. 5, which produces out of phase sound that can be reproduced at the speakers 706.

FIG. 7 depicts an embodiment having one ANC unit, a six-channel pre-amplifier, six speakers, and six total microphones. This embodiment contemplates an upper cancelable frequency of about 700 Hz. This embodiment is suitably configured for use with an inner frame size of 600 mm (height) by 760 mm (width).

In this embodiment, there are three sets of speakers 706 placed 200 mm apart, facing the other three sets of speakers 706 placed in the opposite direction 760 mm away facing each other. The distances between (a) the error correction microphones 704 themselves, (b) the error correction microphones 704 and the speakers 706, and (c) between the speakers 706 themselves, are strategically set to be not more than 240 mm apart to produce a significant effect of the noise cancellation. 240 mm is approximately half of the wavelength of a 700 Hz signal (485 mm). In general, the maximum frequency range to be able to be cancelled by ANC is when such microphones and speakers are placed in the distances of half of the wavelength of the target maximum frequency. If the system has the microphones and the speakers placed more than 240 mm apart, the system becomes economical because it uses less of such components per area, but it will not be able to effectively cancel frequencies up to 700 Hz. In return, if the microphones and the speakers are placed more dense and less than 240 mm apart, the system can control higher frequencies, however, based on today's market requirements and cost of the components it will become economically unfeasible. Also, the less space between the components, the less air and light comes through the system and the less aesthetic appearance for the solution to be placed in the open air environment.

Once the out of phase noise is reproduced by the six speakers 706 independently, the error correction microphones 704 (which are scattered but strategically located as shown in FIG. 7) detect the sound levels of the delta between the original noise level reached to this space and the out-of-phase signals output from the speakers 706. Such plus or minus (too little cancellation, or too much cancellation signals) information is continuously fed back to the active noise cancellation unit or units for continuous adaptation and corrections. The dotted line circles in FIG. 7 are for reference only to show the distances between the error correction microphones 704 themselves, or between the error correction microphones 704 and the speakers 706 (the diameter of 240 mm). In practice, the error correction microphones 704 are placed between the open-air speakers 706 in a manner that compensates for the distance between the speakers 706.

In the practical embodiment, the width between the sets of speakers 706 can be narrower, or wider than the 760 mm as illustrated in FIG. 7, as far as all of the distances (a) between the error correction microphones 704 themselves, (b) the distance between the error correction microphones 704 and the speakers 706, and (c) the speakers 706 themselves, are kept within 240 mm. 240 mm is a practical target which is half of the wavelength of the 700 Hz sound wave. 700 Hz is a practical upper limit of sound frequency which can be controlled economically and effectively by active noise cancellation. One may place speakers and microphones closer to each other to try to reduce higher frequency noise, however, the effect and the cost do not match.

As described above, one particular feature of this system is that all of the speakers do not have to be within such upper limit distance, but utilizing placement of the error correction microphones in such upper limit distances and their feed back functions, some of the speakers can be placed more than the upper limit intervals. In the above example, some speakers are placed 760 mm or more apart, not always within 240 mm apart to each other.

FIG. 8 is a schematic representation of an embodiment of a system 800 having a plurality of active noise cancellation units assembled to form a protected area. System 800 generally employs an open frame structure 801 (which may include any number of vertical, horizontal, or other frame elements) that serves as the mounting structure for the system components. In operation, open frame structure 801 is positioned such that it separates the noise source side of the environment from the quiet side of the environment. Here, open frame structure 801 may be configured as an open-air dividing wall. Open frame structure 801 (and the other open frame structures described herein) preferably includes openings formed therein that allow air to flow through the open frame structure 801. Alternatively or additionally, open frame structure 801 (and the other open frame structures described herein) may be suitably configured to allow light to pass unobstructed between the noise source side and the quiet side of the open frame structure 801.

FIG. 8 shows an example of implementation with multiple active noise cancellation units placed next to another and assembled together to form a 10×10×7 coverage of noise reduction area. The speaker boxes are designed so that they look like pillars of the patio covers. The active noise cancellation units are placed on the top as well as on the sides of the open air area creating an “open air patio covered area.” In this particular example, 48 active noise cancellation units are used, which in this example correspond to 288 speakers, 48 noise cancel microphones, and 240 error correction microphones. In a practical implementation, depending on the requirement of the size of space to reduce the noise, active noise cancellation units can be assembled and built by the increments of 760 mm×600 mm in size.

Because of continuous reduction in cost of electronics components including the speaker drivers, speaker boxes, DSP (digital signal processors), amplifiers, microphones, and other electronic components, even with the use of many components, the system is cost effective to be able to price the products at a reasonable level. Also, cost effective DSPs allow control of multiple active noise cancellation adaptation and filtering and drive and control multiple speakers and microphones.

In addition, the acoustic blades 802 shown on the side and the top left of the structure in FIG. 8, using porous absorptive materials, are strategically placed to reduce high frequency noise. Because of economical reasons, ANC is designed to reduce noise having frequencies of up to 700 Hz. The acoustic blades 802 instead are designed to absorb and reduce diffraction of noise in the higher frequencies over 700 Hz. In this regard, the acoustic blades 802 preferably include sound-absorbing material having properties and characteristics for reducing the anticipated noise frequencies. For an example, as described in connection with FIG. 1, 1 kHz noise can be reduced by about 12 dB with 10 cm of more travel path with such acoustic blades 802. Thus, a system as described herein utilizes a combination of active noise cancellation and acoustic noise absorptive materials to reduce a wide range of noise frequencies.

Note: In this example, there are 48 ANC units (9×4+12), corresponding to 9 units for each side and 12 units on the top. In FIG. 8, the ANC units 804 are depicted as circular items surrounded by a box. For each side, there are 54 speakers (6×9 ANC units), and 72 speakers on the top (6×12 ANC units) for a total of 288 speakers. The height of the system is 2100 mm (3×600 mm speaker box height+3×100 mm frame), and the width is 2880 mm (3×760 mm+6×100 mm speaker box width).

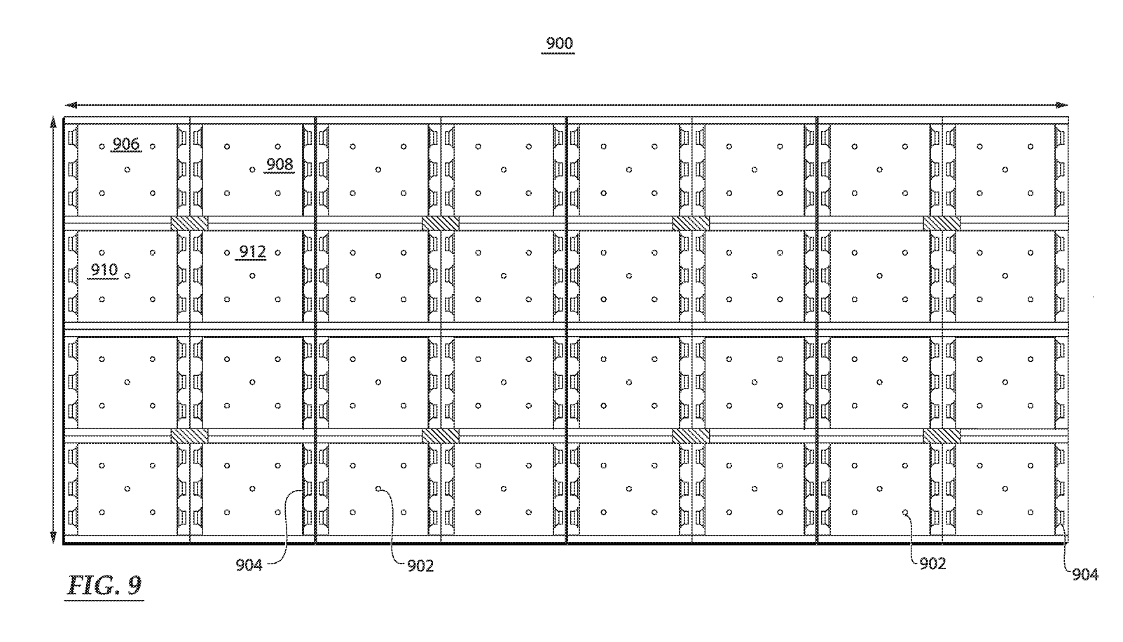

FIG. 9 is a schematic representation of another embodiment of a system 900 having a plurality of active noise cancellation units. For this particular application, 32 ANC units are arranged as a panel wall. This panel wall allows air flow and light passage, but reduces unwanted noise by a factor of 50% or more. In this example there are 32 noise collection microphones, 160 noise correction microphones, and 192 noise cancellation speaker drivers. This panel will be placed such that the noise collection microphones are facing towards the noise source, with an open air environment on the other side of the panel.

This panel can also be placed on the surfaces of a roof and a wall of a building close to an airport and other noise sources to reduce low frequency noise going though the existing structures. Because lower frequency sound tends to travel through rigid walls and window glass, the application to such building structure reduces the cost for insulation and provides further reduction of low frequency noise.

In FIG. 9, the error correction microphones 902 are depicted as small dots “floating” within the open spaces defined between the horizontal structures and the vertical structures (the vertical structures serve as mounts for the speakers). The speakers 904 are depicted from the side in FIG. 9. The eight cross-hatched areas in FIG. 9 represent locations for the noise collection microphones, the ANC units, and power circuitry. Each location may service four ANC units, 20 error correction microphones 902, four noise collection microphones, and 24 speakers 904. For example, the upper left location may support the system components for the four adjacent areas (reference numbers 906, 908, 910, and 912). This configuration is desirable because it results in relatively short lines and signal paths between the microphones and the ANC units. In practice, system power may be provisioned through a main power line that enters system 900 from the top, bottom, or one of the side frame elements.

One example of system 900 has an overall width of 7840 mm and an overall height of 2550 mm. Of course, the overall dimensions may vary to accommodate more or less ANC subsystems and to suit the needs of the particular installation.

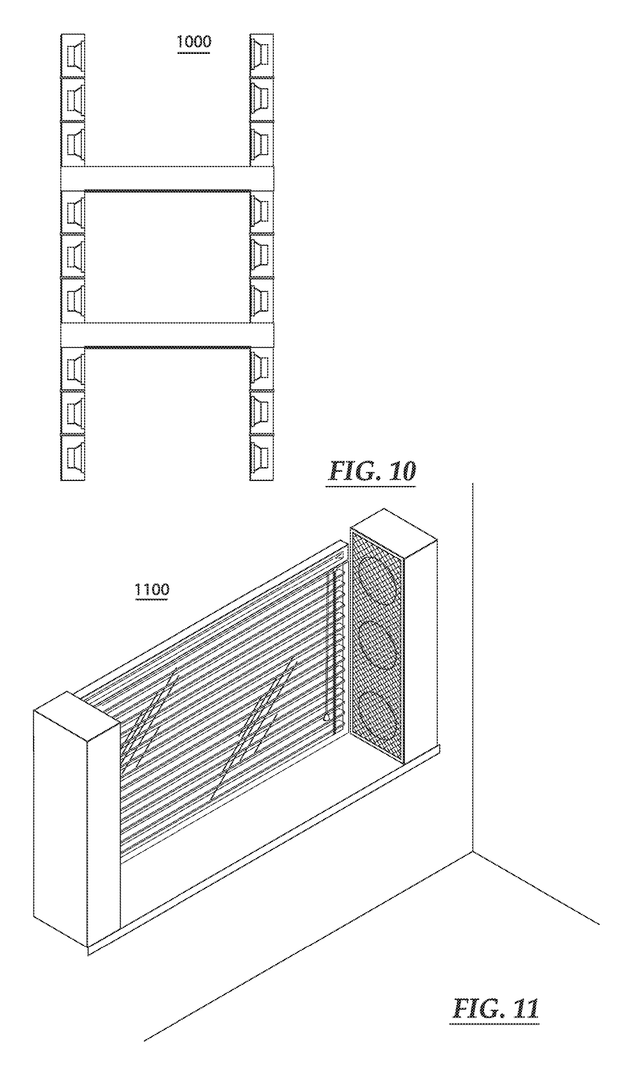

FIG. 10 is a schematic front view of an embodiment of a door/window noise cancellation unit 1000, and FIG. 11 is a perspective view of portion of a window noise cancellation unit 1100. FIG. 10 and FIG. 11 show an example of a shutter noise cancellation unit suitable for use in a door or window installation. In noise cancellation unit 1000, three active noise cancellation units are used which corresponds to 18 speakers, three noise collection microphones (not shown), and 15 error correction microphones (not shown). The active noise cancellation units reduce noise having frequencies up to 700 Hz.

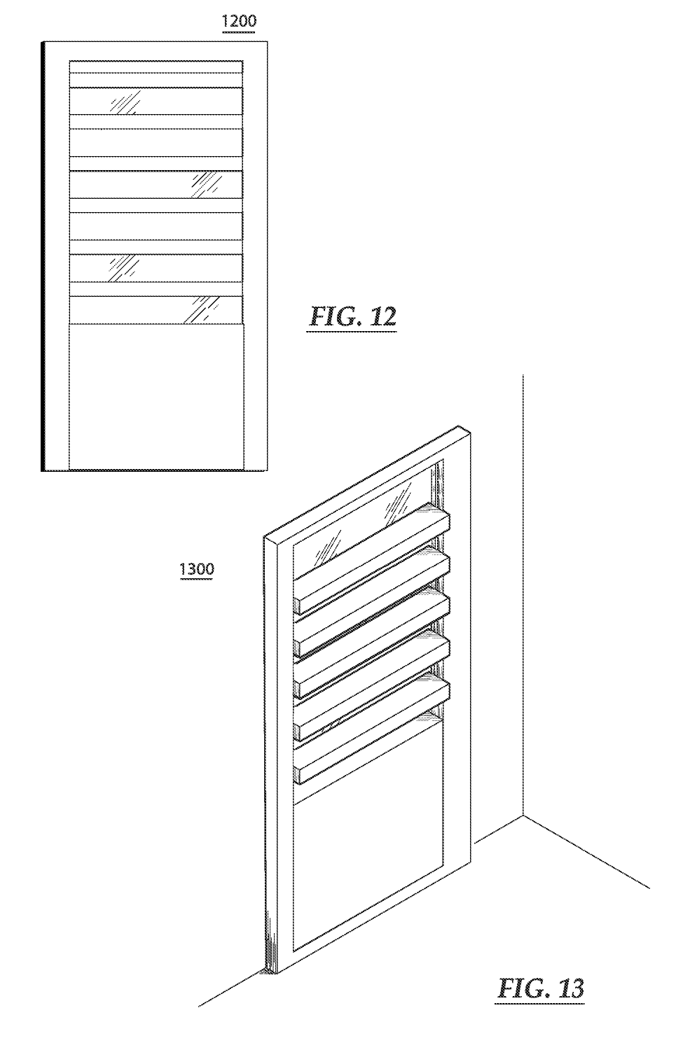

In practice, acoustic blades may be employed to reduce noise having frequencies higher than 700 Hz. In this regard, FIG. 12 is a schematic front view of an embodiment of a door/window noise cancellation unit 1200 that utilizes shutters, and FIG. 13 is a perspective view of a portion of a window noise cancellation unit 1300 that utilizes shutters. Notably, the shutter designs depicted in FIG. 12 and FIG. 13 can be combined with the ANC designs depicted in FIG. 10 and FIG. 11 and with any of the ANC configurations described above.

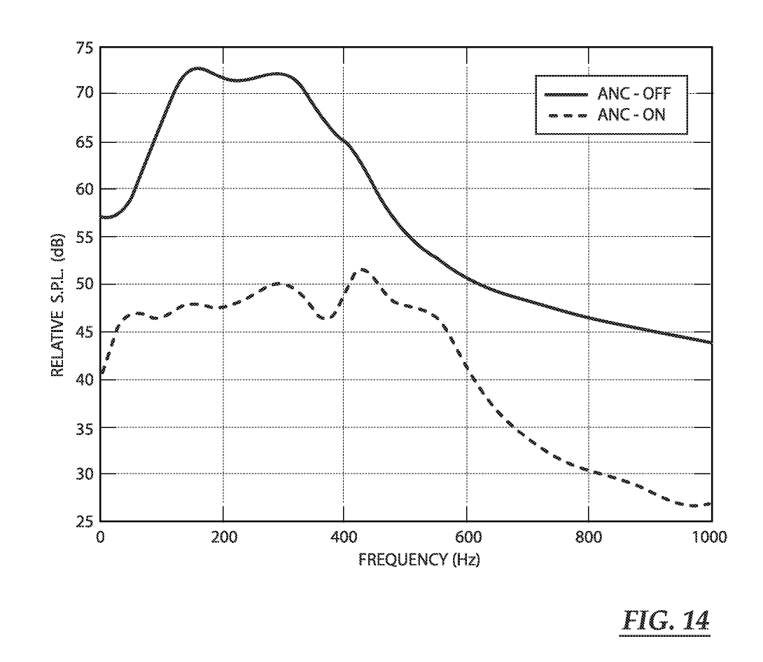

FIG. 14 is a graph of a typical noise characteristic of a jet aircraft flying at 500-1000 feet altitude, both before and after active noise cancellation using a system as described herein. The horizontal axis represents frequency and the vertical axis represents relative sound pressure level. The solid graph represents the original noise characteristic and the dashed graph represents the noise characteristic with active noise cancellation applied. A system according to the invention reduces the low frequency jet engine noise with the active noise cancellation up to 700 Hz, and the higher frequency noise with the acoustic blades over 700 Hz. Notably, the noise reduction in the low frequency band is significant—more than 20 dB for certain low frequencies.

The system described herein allows cancellation and reduction of background noise such as highway traffic noise, airplane noise, industrial noise, air conditioner and home equipment noise, office noise, and other noise in the open-air environment, as an installed device.

While at least one example embodiment has been presented in the foregoing detailed description, it should be appreciated that a vast number of variations exist. It should also be appreciated that the example embodiment or embodiments described herein are not intended to limit the scope, applicability, or configuration of the invention in any way. Rather, the foregoing detailed description will provide those skilled in the art with a convenient road map for implementing the described embodiment or embodiments. It should be understood that various changes can be made in the function and arrangement of elements without departing from the scope of the invention, where the scope of the invention is defined by the claims, which includes known equivalents and foreseeable equivalents at the time of filing this patent application.

What is claimed is:

1. A noise cancellation system for open air noise reduction, the system comprising:
a plurality of open-air speakers;
a noise collection microphone located proximate to the open-air speakers, the noise collection microphone being configured to obtain a noise signal;
a plurality of error correction microphones configured to detect a difference between an original noise signal and a corresponding out-of-phase noise signal generated by the plurality of open-air speakers; and
a processing architecture configured to generate a noise cancellation signal based upon the original noise signal and to continuously adapt to optimize the noise cancellation signal.

2. A system according to claim 1, the open-air speakers and the plurality of error correction microphones being configured and positioned based upon a frequency to be controlled.

3. A system according to claim 1, the plurality of error correction microphones being configured and positioned in accordance with anticipated frequencies of the original noise signal, and the plurality of error correction microphones being placed between the plurality of open-air speakers to compensate for the distance between the speakers.

4. A system according to claim 1, further comprising acoustic blades comprising sound absorbing material, the acoustic blades being located proximate to the plurality of open-air speakers, and the acoustic blades being configured to reduce higher frequency noise signal components.

5. A noise cancellation system comprising:
a sound reduction space having a noise source side and a quiet side;
a noise collection microphone located on the noise source side, and being configured to obtain a noise signal;
a plurality of open-air speakers located on the quiet side, and being configured to generate noise cancellation sound waves;
a plurality of error correction microphones located on the quiet side, and being configured to obtain an error correction signal; and
a processing architecture configured to generate noise cancellation signals based upon the noise signal and based upon the error correction signal.

6. A system according to claim 5, further comprising an open frame structure configured to separate the noise source side from the quiet side, wherein:
the plurality of open-air speakers are mounted to the open frame structure; and
the open frame structure is configured to allow air to flow between the noise source side and the quiet side.

7. A system according to claim 6, wherein the open frame structure forms an open-air dividing wall.

8. A system according to claim 6, wherein the open frame structure forms an open-air canopy.

9. A system according to claim 6, wherein the open frame structure forms an open-air enclosure for a protected area on the quiet side.

10. A system according to claim 5, further comprising an open frame structure configured to separate the noise source side from the quiet side, wherein:
the plurality of open-air speakers are mounted to the open frame structure; and
the open frame structure is configured to allow light to pass unobstructed between the noise source side and the quiet side.

11. A system according to claim 5, further comprising a plurality of acoustic blades, wherein each of the plurality of acoustic blades is configured to reduce higher frequency components of the noise signal.

12. A system according to claim 5, further comprising a plurality of acoustic blades, wherein each of the plurality of acoustic blades is configured to influence diffraction of the noise signal such that at least a portion of the noise signal diffracts away from the quiet side.

13. A noise cancellation system for open air noise reduction, the system comprising:
an open frame structure having openings formed therein that allow air to flow through the open frame structure;
a plurality of noise collection microphones located proximate to the open frame structure, each of the noise collection microphones being configured to detect sound waves on a noise source side of the open frame structure;
a plurality of open-air speakers mounted to the open frame structure, each of the open-air speakers being configured to generate noise cancellation sound waves on a quiet side of the open frame structure; and
at least one active noise cancellation unit mounted to the open frame structure, the at least one active noise cancellation unit being configured to generate noise cancellation signals in response to the detected sound waves, wherein the noise cancellation signals influence characteristics of the noise cancellation sound waves generated by at least one of the open-air speakers.
